(19) United States
(12) Patent Application Publication
(10) Pub. No.: US 2017/0213002 A1
(43) Pub. Date: Jul. 27, 2017

(54) SAFETY-DRIVEN ARCHITECTURE FOR IMPLANTABLE AND WEARABLE MEDICAL DEVICES

(71) Applicants: Niraj K. Jha, Princeton, NJ (US); Younghyun Kim, West Lafayette, IN (US); Vijay Raghunathan, West Lafayette, IN (US); Anand Raghunathan, West Lafayette, IN (US)

(72) Inventors: Niraj K. Jha, Princeton, NJ (US); Younghyun Kim, West Lafayette, IN (US); Vijay Raghunathan, West Lafayette, IN (US); Anand Raghunathan, West Lafayette, IN (US)

(73) Assignee: The Trustees of Princeton University, Princeton, NJ (US)

(21) Appl. No.: 15/416,648

(22) Filed: Jan. 26, 2017

Related U.S. Application Data
(60) Provisional application No. 62/287,757, filed on Jan. 27, 2016.

(51) Int. Cl. G06F 19/00; A61B 5/00; G08B 21/02; A61N 1/37; A61M 5/172; G06Q 50/26; A61B 5/145

(52) U.S. Cl. CPC G06F 19/3406; G06Q 50/265; A61B 5/4836; A61B 5/4839; A61B 5/7282; A61B 5/14532; A61N 1/3704; A61M 5/1723; G08B 21/02; A61M 2005/1726

(57) ABSTRACT

An implantable/wearable medical device is configured for use with a plurality of sensors. The device includes a host microcontroller, a safety coprocessor and an actuator. The host microcontroller is configured to receive physiological data from the sensors and generate actuator commands for the actuator. The host microcontroller is configured to generate program state data for transmission to the safety coprocessor. The safety coprocessor is configured to receive the physiological data from the sensors and I/O access data and the program state information from the host microcontroller and determine whether there is a safety rule violation. The safety coprocessor is also configured to issue the actuator command to the actuator if no safety rule violation is detected. The safety coprocessor is also configured to initiate safety procedures if a safety rule violation is detected.

### CROSS-REFERENCE TO PRIOR FILED APPLICATIONS

This application claims priority to U.S. provisional application 62/287,757, filed Jan. 27, 2016, which is incorporated herein in its entirety.

### STATEMENT REGARDING FEDERALLY SPONSORED RESEARCH OR DEVELOPMENT

This invention was made with government support under Grant No. CNS-1219570 awarded by the National Science Foundation. The government has certain rights in the invention

### FIELD OF THE INVENTION

The present disclosure generally relates to implantable and wearable medical devices and in more particular implantable and wearable medical devices with hardware configurations that address security vulnerabilities.

### BACKGROUND

Implantable and wearable medical devices (IWMDs) have greatly improved therapeutic outcomes and quality-of-life for patients by enabling continuous and autonomous management of a wide range of medical conditions, including diabetes, cardiac arrhythmia, epilepsy, and gastrointestinal conditions. The use of IWMDs has been growing steadily over the past decade. Modern IWMDs are embedded computing platforms that execute sophisticated algorithms for detection and response in real-time. They also provide wireless connectivity for post-deployment diagnostics, tuning of therapy, and access to medical data by the patient and healthcare provider.

Unfortunately, these advances are accompanied by higher hardware/software (HW/SW) complexity, leading to an increase in unreliability as well as security vulnerabilities. Complex IWMD firmware often contains bugs that cannot be completely eliminated at design time. A recent research study attributed 64.3% of medical device recalls issued by the U.S. Food and Drug Administration (FDA) between 2006 and 2011 to SW-related issues. Researchers have also demonstrated how wireless connectivity can be exploited to remotely hijack IWMDs and cause unsafe, even life-threatening, behavior.

Regardless of cause—firmware bugs, malicious attacks or even user errors—unsafe operation of IWMDs is an utmost concern of patients. We, therefore, propose to enhance the IWMD's HW/SW to identify unsafe IWMD operations and even prevent them before they can have an adverse impact on the patient. There are four main challenges involved in designing such a safety mechanism for IWMDs. First, whether a given IWMD behavior is safe or unsafe is context-dependent. For example, in the case of an insulin pump, a bolus insulin dose can be determined to be safe or unsafe depending on the patient's blood glucose (BG) level; similarly, a high-voltage defibrillation pulse generated by an implantable cardioverter defibrillator (ICD) can be deemed unsafe if it is produced shortly after a wireless packet is processed and the sensed heartbeat is normal. Thus, high-level context awareness is essential for accurate decision-making regarding safety. Second, unsafe operations should be identified and blocked in a proactive manner before they are actually performed because IWMD operation may be irreversible once they take place (e.g., infused insulin cannot be removed). Third, it is desirable that safety checking be performed in a computing environment that is isolated from the normal computing environment in the IWMD to prevent contamination from failures and security attacks. A modular design will also facilitate integration into existing IWMDs that are designed under extremely conservative development and regulatory processes. Finally, due to the stringent energy constraints imposed on battery-powered IWMDs, the energy overheads imposed by the checking mechanism should be kept as low as possible.

### SUMMARY OF THE INVENTION

An implantable/wearable medical device is configured for use with a plurality of sensors. The device includes a host microcontroller, a safety coprocessor and an actuator. The host microcontroller is configured to receive physiological data from the sensors and generate actuator commands for the actuator. The host microcontroller is configured to generate program state data for transmission to the safety coprocessor. The safety coprocessor is configured to receive the physiological data from the sensors and I/O access data and the program state information from the host microcontroller and determine whether there is a safety rule violation. The safety coprocessor is also configured to issue the actuator command to the actuator if no safety rule violation is detected. The safety coprocessor is also configured to initiate safety procedures if a safety rule violation is detected.

The safety coprocessor may be configured to perform safety rule checking based on state transition rules, I/O access rules and physiological rules. The state transition rules may be based on the host microcontroller program state. The I/O access rules may be based on access to I/O components. The physiological rules may be based on physiological data received from the sensors. The physiological rules may be based on a time lapse. The safety coprocessor may be configured to communicate with a user interface to generate an alarm after a safety rule violation is detected. The safety coprocessor may be configured to reset the host microcontroller after a safety rule violation is detected.

The safety coprocessor may be configured with a safety rule evaluation engine, a violation response engine and a steering logic engine. The safety rule evaluation engine may be configured to receive the program state data from the host microcontroller, sensor data from sensors and actuator commands from the host microcontroller, the safety rule evaluation engine being configured to detect a safety rule violation and generate a rule violation status output and cut off output. The steering logic engine may be configured to receive the cut off output from the safety rule evaluation engine and if no safety rule violation is detected, the actuator command is routed to the actuator, if a safety rule violation is detected the steering logic engine blocks the actuator command or sensor data from reaching the host microcontroller.

A method for detecting a safety rule violation in an implantable/wearable medical device configured for use with a plurality of sensors is also disclosed. A host microcontroller, a safety coprocessor and an actuator are provided. The host microcontroller is configured to receive physiological data from the sensors and generate actuator commands for the actuator. The host microcontroller is also configured to generate program state data for transmission to the safety coprocessor. The safety coprocessor is configured to receive the physiological data from the sensors and I/O access data and the program state information from the host microcontroller and determine whether there is a safety rule violation. The safety coprocessor is configured to issue the actuator command to the actuator if no safety rule violation is detected. The safety coprocessor is also configured to initiate safety procedures if a safety rule violation is detected.

The safety coprocessor may be configured to perform safety rule checking based on state transition rules, I/O access rules and physiological rules. The state transition rules may be based on the host microcontroller program state. The I/O access rules may be based on access to I/O components. The physiological rules may be based on physiological data received from the sensors. The physiological rules may be based on a time lapse. The safety coprocessor may be configured to communicate with a user interface to generate an alarm after a safety rule violation is detected. The safety coprocessor may be configured to reset the host microcontroller after a safety rule violation is detected.

The safety coprocessor may be is configured with a safety rule evaluation engine, a violation response engine and a steering logic engine. The safety rule evaluation engine may be configured to receive the program state data from the host microcontroller, sensor data from sensors and actuator commands from the host microcontroller, the safety rule evaluation engine being configured to detect a safety rule violation and generate a rule violation status output and cut off output. The steering logic engine may be configured to receive the cut off output from the safety rule evaluation engine and if no safety rule violation is detected, the actuator command is routed to the actuator, if a safety rule violation is detected the steering logic engine blocks the actuator command or sensor data from reaching the host microcontroller.

### BRIEF DESCRIPTION OF THE FIGURES

FIG. 1A is a block diagram of an IWMD architecture with a safety coprocessor;

FIG. 1B is a block diagram of an IWMD architecture illustrating the flow of communications between the host microcontroller and safety coprocessor;

FIG. 2A shows how BG levels are controlled by an insulin pump therapy system;

FIG. 2B shows a simplified program state diagram of an insulin pump;

FIG. 3 shows safety rule checking flow of an insulin pump;

FIG. 4 shows a prototype insulin pump system;

FIG. 5 shows a prototype insulin pump and human body model simulator integrated for safety rule development and evaluation;

FIG. 6 shows the communication waveform from receiving an RF packet to issuing a command to the pump driver;

FIG. 7 shows a buffer overflow that triggers the micro pump to infuse the maximum amount of insulin; and

FIG. 8 shows BG level changes (a) in a normal case, (b) when dinner and bolus insulin are skipped, (c) when bolus insulin is infused without taking a meal, and (d) when skipped meal is taken after a safety warning is raised.

To facilitate understanding, identical reference numerals have been used, where possible, to designate identical elements that are common to the figures.

### DETAILED DESCRIPTION

Disclosed herein is a hardware/software (HW/SW) architecture that employs a safety coprocessor to address the aforementioned challenges for IWMDs. This coprocessor is integrated into the IWMD in such a way that it has full visibility of the I/O activities performed by the host microcontroller, thereby enabling it to monitor a range of useful information, including the control flow of the device firmware, sensor inputs received, actuator commands generated, and packets received from the wireless network interface. The safety coprocessor uses this information in a decision process to evaluate the safety of IWMD operations. In order to ensure that unsafe operations are prevented, all actuator commands issued by the host microcontroller need to be validated by the safety coprocessor before they reach the appropriate peripherals. In effect, the coprocessor acts as the last line of defense for preventing unsafe operations from impacting the patient. To enable flexible and robust safety checking, we realize the safety coprocessor using a low-power microcontroller that is physically isolated from the host microcontroller used in the IWMD. The proposed architecture has been implemented to demonstrate its effectiveness with the help of a prototype insulin pump. While the concept of a coprocessor for reliability and security have been explored in the context of other computing systems, the applicants are unaware of any efforts aimed at employing such an architecture in IWMDs.

Some of the various aspects disclosed herein include:

A HW/SW architecture for improved IWMD safety. It is based on a physically isolated low-power safety coprocessor that makes decisions about the safety of IWMD operations and blocks them if they are determined to be unsafe. Safety checking is based on the physiological status of the patient and the context of the behavior inferred from the activities of the host microcontroller.

This disclosure also includes a design and implementation of a safety-enhanced IWMD controller board based on the proposed architecture. In this example a Cortex M4 embedded microcontroller is used as the host microcontroller that executes the device firmware and a Cortex M0+ low-power microcontroller is used as the safety coprocessor. Safety rule checking is implemented based on the control flow of the device firmware, I/O activities of the host microcontroller, and the patient's inferred physiological status, to detect unsafe behavior.

This disclosure also includes a case study in which a prototype insulin pump is implemented using the proposed IWMD controller board integrated with a physiological simulator running on a PC. The simulator incorporates a human body model to emulate the outcomes of IWMD operations. The effectiveness of the proposed architecture is demonstrated against various practical safety risks and evaluate its overheads.

Unsafe Operation of IWMDs

IWMDs may exhibit unsafe operation due to user errors, SW bugs or malicious attacks. Previous studies have shown that each of these causes may be significant and do occur in practice. For example, a study of insulin pump malfunctions reported that user errors contribute to a significant portion of adverse events. Another recent study classified safety advisories issued by the FDA and pointed to SW bugs as a major source of safety risks in medical devices. For example, a firmware malfunction in a glucose meter was found to result in unexpected behaviors at BG levels of 1,024 mg/dL or higher. Recent years have also seen security attacks on IWMDs being transformed from a remote possibility to an immediate concern. Due to stringent energy constraints and unique usage model of IWMDs (requiring unhindered access in an emergency), their wireless channels are often designed without strong cryptography. These vulnerabilities have been exploited to demonstrate successful security attacks by researchers and security practitioners. Unsurprisingly, medical device security is increasingly becoming a focus of regulatory concern.

While it is easy to provide such examples of unsafe operation, distinguishing between safe and unsafe behavior in a general, yet precise, manner is far from straightforward. A key observation in this regard is that combining multiple sources of information enables more effective and discriminative safety checking. For example, the safety of a bolus insulin infusion command in an insulin pump depends on the current BG level of the patient and time interval since previous infusion (the operation may be highly beneficial if the patient's BG level is high or rapidly trending upwards, but harmful if the patient's BG level is already low). At the same time, the control path taken within the device firmware can be used to infer whether an insulin infusion command was caused by a command packet received over the wireless channel or was autonomously issued by the pump based on the sensed BG level. Therefore, combining these sources of information can form the basis for more robust and accurate determination of the safety of IWMD operations.

Related Work

The past few years have witnessed great interest in the reliability and security of IWMDs. Due to the increasing complexity of IWMD firmware, empirical testing falls far short of guaranteed correctness. Formal methods have been explored in this context, but their practical adoption is impeded by legacy HW/SW, for which formal models do not exist, and the well-known state-space explosion problem. Run-time monitoring techniques that look for anomalies in program control flow can detect SW bugs or vulnerability exploits. However, these techniques do not take the physiological context of IWMD operations into account, and are, hence, neither complete nor able to make precise decisions regarding safety.

Various techniques have been proposed to enable cryptographic protection of the wireless communication channel in IWMDs, including exchanging secret cryptographic keys over a physically secure non-RF side channel and generating keys from physiological signals. To protect legacy IWMDs that do not employ cryptography or to offload power-hungry security functions from IWMDs, external devices dedicated to IWMD protection have been proposed. These devices protect the IWMD communication channel from security attacks, but are not capable of detecting device malfunctions or vulnerabilities that originate from within the IWMD.

To sum up, in spite of substantial prior research efforts, there remains a gap between the need for safety assurance mechanisms in IWMDs and the present state-of-the-art. Specifically, there is a need for a safety assurance mechanism that (i) is capable of detecting both device malfunctions and security attacks, (ii) makes precise decisions about safety with awareness of relevant physiological parameters, and (iii) is easy to integrate into the HW/SW of existing IWMDs.

Hardware/Software Architecture

This section discloses the important requirements that an IWMD safety assurance mechanism must satisfy. A HW/SW architecture is disclosed with a safety coprocessor and detail its operation, including safety rule checking performed by the coprocessor to detect and prevent various unsafe operations.

Requirements for IWMD Safety Assurance

Based on the aforementioned observation of safety challenges, we suggest that any safety assurance mechanism for IWMDs should satisfy the following key requirements:

Visibility—the ability to obtain necessary information about the operational state of the IWMD in order to make inferences about safety. Safety of an IWMD operation is primarily determined by the current physiological status of the patient as well as how the device performs the operation. Therefore, to make accurate decisions, the safety mechanism should have visibility into both the extrinsic state (i.e., the patient's physiological parameters sensed by the IWMD) and the intrinsic state of the IWMD itself (i.e., the host microcontroller's activities).

Accessibility—the safety mechanism should be able to take control of appropriate points in the IWMD in a timely manner. Once an unsafe operation is detected, it must be blocked before it actually acts on the patient's body. In most cases, IWMDs inject either a drug or an electrical signal into the patient's body through actuators. Therefore, the safety mechanism should be able to stop unsafe actuator commands before they are executed.

Isolation—the safety assurance mechanism should be isolated from the medical functionality itself. Separating safety functionality from complex medical functionality provides the benefits of i) minimal modification of the existing medical functionality, ii) ease of verification of the safety assurances at design time, and iii) prevention of failure propagation to the safety-critical functionality. However, care must be taken to balance isolation with the previous two requirements because an isolated safety mechanism may have limited visibility and accessibility.

Architecture with a Safety Coprocessor

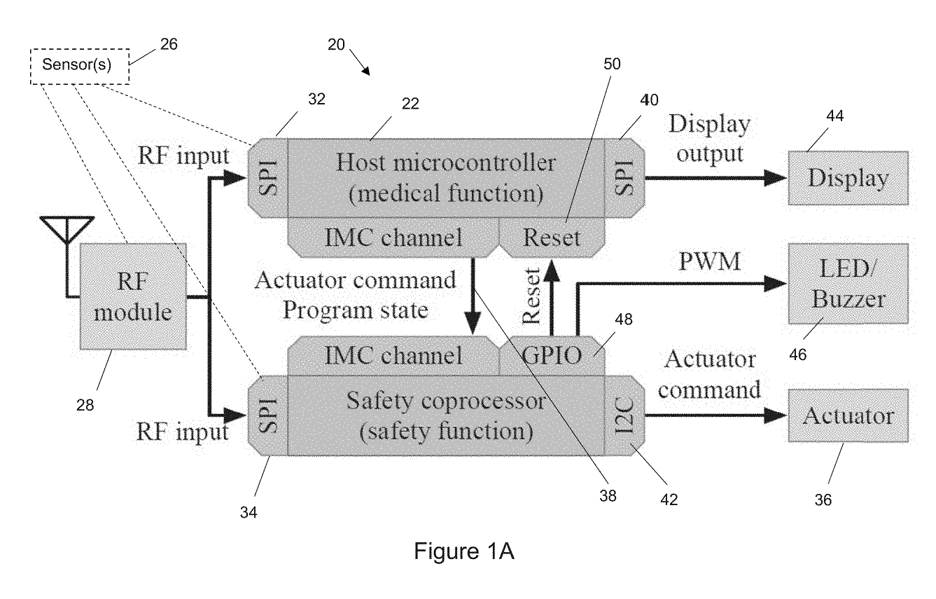

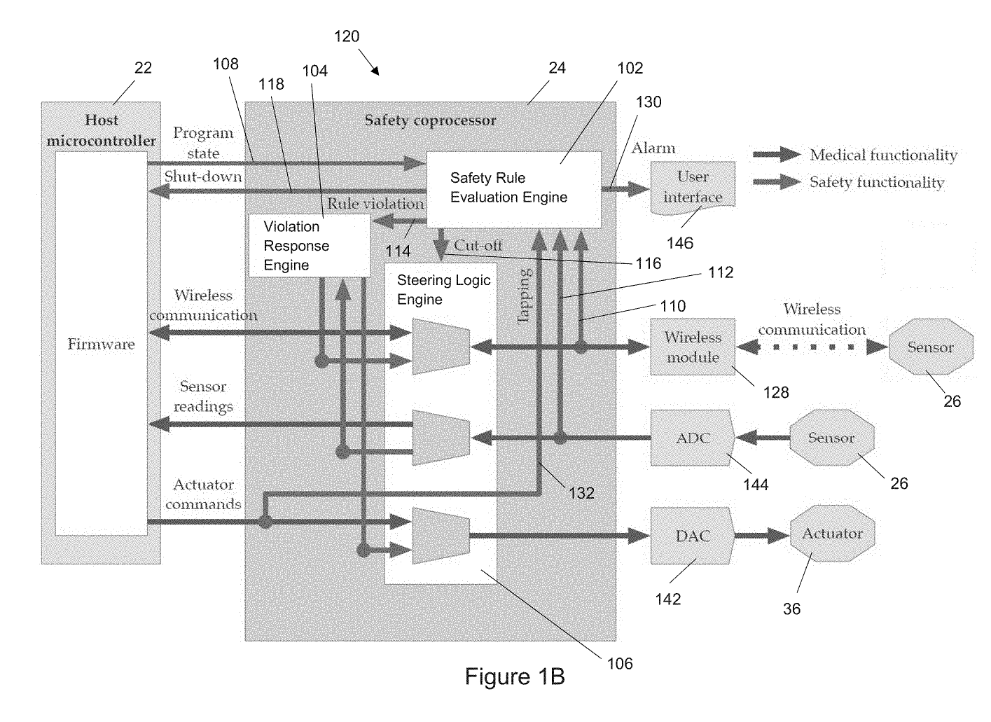

This section discloses details of the architecture that addresses the above-mentioned requirements. FIG. 1A is a block diagram of an IWMD architecture 20 with a host microcontroller 22 and a safety coprocessor 24.

One or more sensors shown generally by reference number 26 are coupled to the host microcontroller 22 and a safety coprocessor 24. The sensors may be coupled to the host microcontroller 22 and a safety coprocessor 24 via conventional techniques such as wired and wireless connections shown generally by the dashed lines to the RF module 28 and interfaces 32 and 34 (in this example serial to parallel interfaces).

In general, the medical functionality is executed by the host microcontroller 22. The host microcontroller 22 receives sensor data from the sensors 26. The host microcontroller 22 then generates and issues actuator commands for the actuator 36. In a conventional design the actuator commands would be communicated to the actuator directly from the host microcontroller. Safety is addressed in this architecture through the safety coprocessor 24. The main role of the safety coprocessor 24 is to monitor various device operations, determine if the host microcontroller 22 or other system component are operating safely and block any actions by the host microcontroller 22 that are determined to be unsafe.

The safety coprocessor 24 has high visibility into both the patient's physiological state as well as device behavior. First, it monitors the sensor data received from the sensors 26 by snooping on the on-board communication channel. In IWMDs where the sensor is integrated in a single device, this is done by snooping the sensor-to-host microcontroller communication channel. The safety coprocessor 24 only listens to the communication in a passive manner and does not interfere unless an unsafe operation is detected. At the same time, it also monitors the program state of the firmware running on the host microcontroller over a dedicated communication channel called the inter-microcontroller communication (IMC) channel shown generally by reference number 38. The host microcontroller generates messages whenever the program state changes (e.g., when entering and exiting critical functions) so that the safety processor can keep track of it. These state change messages are communicated from the host microcontroller 22 to the safety coprocessor 24 via the IMC channel 38.

Unlike sensor inputs that are only passively monitored, actuator commands are intercepted in between the host microcontroller 22 and actuator by the safety coprocessor 24 via the IMC channel 38. The intercepted actuator commands are inspected by the safety coprocessor 24 and passed on to the actuator 36 only when their safe behavior is confirmed. There is no connection between the host microcontroller 22 and the actuator 36. If the safety coprocessor 24 determines that the command is not safe, it blocks the command and takes necessary actions, as described in herein. This rerouting of actuator commands grants the safety coprocessor 24 the ability to counteract potentially unsafe operation in a timely manner, before it has actual impact. While the host microcontroller 22 no longer has direct control over the actuator, this rerouting is transparent to the host microcontroller. Thus, it requires only minimal HW modifications for existing IWMD designs.

In this example, the host microcontroller 22 is configured with an interface 40 (in this example serial to parallel interface) that may be coupled to external devices via conventional techniques such as a display 44. Similarly, the safety coprocessor 24 is configured with an interface 42 (in this example serial an Inter-Integrated Circuit or I²C interface) that is coupled to the actuator 36. In this example the safety coprocessor 24 is configured with a second interface 48 (in this example a general-purpose input/output (GPIO) interface) for communications of safety related communications. For example the second interface is coupled to the reset circuitry 50 of the host microcontroller 22 so that under certain conditions the host microcontroller 22 can be reset by the safety coprocessor 24. The second interface may also be coupled to a user interface or alarm device such as a display, LED or buzzer, shown generally by block 46, to alert the user that a safety concern has been identified. It should be understood that safety coprocessor interface 42 and second interface 48 may be combined into a single interface depending on the desired hardware implementation.

Even though the safety coprocessor 24 is physically isolated from the host microcontroller 22 and does not actively share any resources, it has the high visibility and accessibility necessary to detect and prevent potentially unsafe operations by monitoring and cutting into off-chip communication channels. The amount of HW modification needed to this architecture is minimal since the safety coprocessor 24 is effectively the only additional hardware and rerouting or tapping off-chip communication buses does not incur significant overheads. The software modifications required in the host microcontroller 24 include generating a unique message that is communicated to the safety coprocessor 24 when the program state changes. This is a minor modification that has negligible influence on the host microcontroller 24 program execution.

FIG. 1B is a block diagram of an IWMD architecture 120 illustrating the flow of communications between the host microcontroller 22 and safety coprocessor 24. The safety coprocessor 24 is configured with a safety rule evaluation engine 102, a violation response engine and a steering logic engine. The safety rule evaluation engine 102 is configured to receive program state information from the host microcontroller 22 as shown by arrow 108 and sensor data from sensors 26 as generally shown by arrows 110 and 112. In this example wired sensors are coupled to the IWMD via analog to digital converter 144 and wireless sensors are coupled to the IWMD via wireless module 128. The safety rule evaluation engine 102 also receives actuator commands from the host microcontroller 22 as shown by arrow 132. This rerouting of actuator commands gives the safety coprocessor the ability to counteract potentially unsafe operation in a timely manner, before it has actual impact. The safety rule evaluation engine 102 determines whether there is a safety rule violation based on the received inputs and generates a rule violation status output and cut off output as generally shown by arrows 114 and 116. The safety rule evaluation engine 102 may also generate an alarm output 130 that is coupled to user interface 146.

The violation response engine 104 receives the rule violation status output 114 and cut off output 116 and generates the appropriate output for the steering logic engine 106. The steering logic engine 106 receives the cut off output 116 and generates an appropriate output. If no safety rule violation is detected, the actuator command is routed to the actuator 36. If a safety rule violation is detected the steering logic engine may block the actuator command and/or sensor reading to/from the host microcontroller 22. [Note—need clarification of the function of the upper two block in the steering logic engine].

Safety Rule Enforcement

The safety coprocessor enables precise decisions about safety based on simple, yet effective, rules. In this section, a detailed explanation of safety rule checking is provided. This provides IWMD developers with a framework that facilitates robust and precise safety rule checking, not to define a complete set of safety rules. An insulin pump is used as an illustrative example.

People with Type 1 diabetes, who have a problem with controlling blood glucose (BG) levels due to their pancreas not producing enough insulin, often rely on an insulin pump to continuously deliver artificial insulin through a catheter under the skin. In this example an insulin pump is used as a reference IWMD model since it is highly interoperable and interactive. However, the disclosed safety architecture can be generally applied to any IWMD, such as a pacemaker or ICD. Modern insulin pumps are wirelessly connected to a continuous glucose meter (CGM) for autonomous BG level monitoring. It can also be manually controlled by the patient using a remote controller. Such high interoperability and frequent user interactions, however, raise reliability and security concerns.

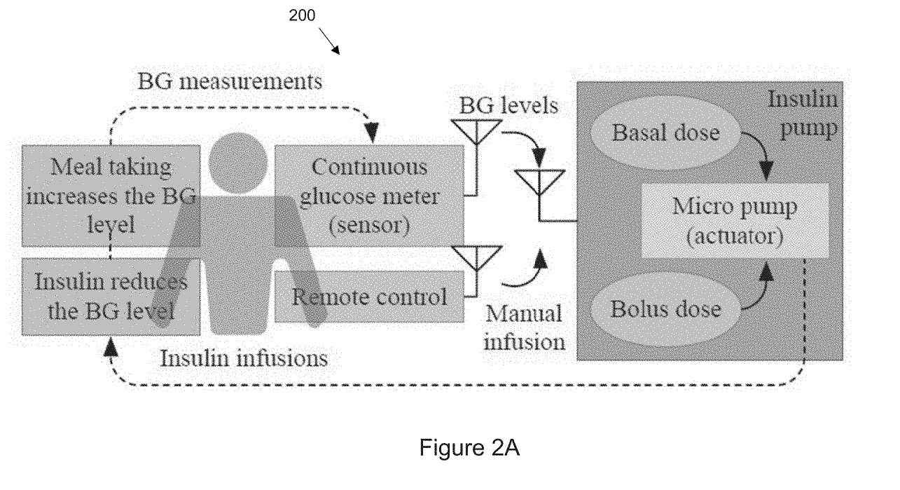

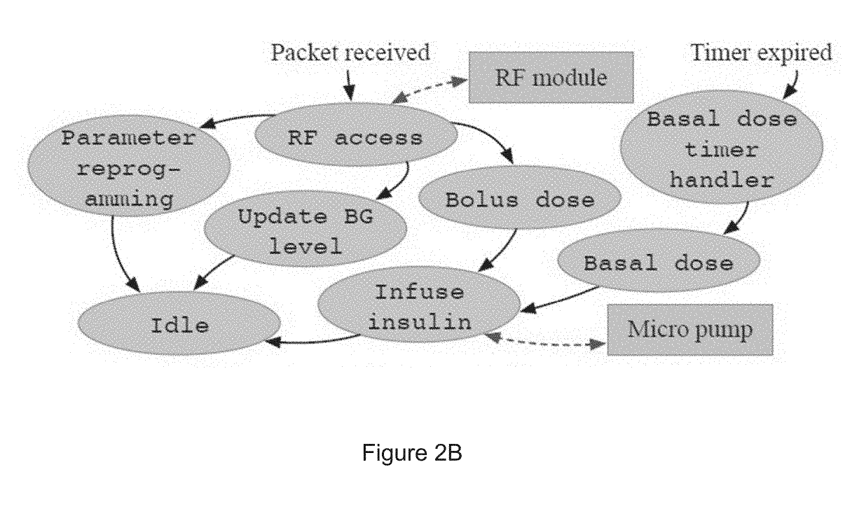

FIG. 2A shows how BG levels are controlled by an insulin pump therapy system 200. The continuous glucose monitor (CGM) periodically measures the BG level and wirelessly transmits the measurements to the insulin pump. The measured BG levels are used to automatically compute the basal dose rate (insulin needed to keep BG levels constant during periods of fasting), and to help the patient compute bolus doses (insulin need to reduce BG levels increased by taking a meal). About 15 to 30 minutes before each meal, to prevent sudden elevations in the BG level, the patient manually requests an infusion of a bolus dose to prevent a high BG level due to the carbohydrate intake from the meal.

Rule Checking by the Safety Coprocessor

The major function of the safety coprocessor is to enforce various safety rules, identify any device activities that violate the rules, and perform follow-up actions. Therefore, specifying robust safety rules based on the expected, safe device behavior is the first and most critical step of the safety assurance mechanism.

FIG. 2B shows a simplified program state diagram of an insulin pump. The ovals denote the program execution states and squares denote the I/O components used by the program. The black solid arrows denote transitions between states and blue dashed arrows denote I/O component access (e.g., sensor, actuator) from each state. The diagram captures expected device behavior at a high level from which safety rules can be derived.

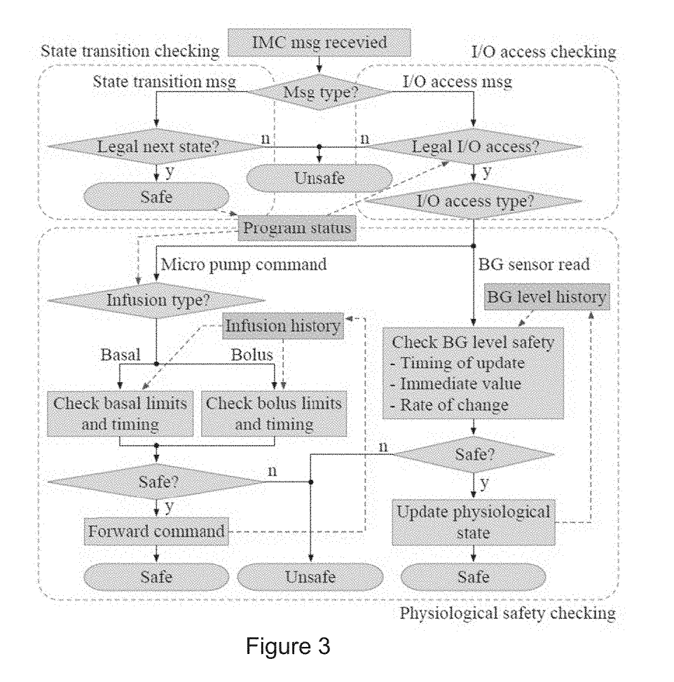

FIG. 3 shows the safety rule checking flow of an insulin pump. The safety coprocessor checks the following general categories of rules: state transition rules, I/O access rules and physiological rules as explained in more detail below.

State transition rules—these rules define the frequency of entering each state, time interval between entering and exiting each state, and legal next states for each state. Checking these rules can detect firmware malfunctions or security attacks that change the execution paths of the program.

I/O access rules—an unexpected access to I/O components suggests potential existence of firmware malfunction or security attack. I/O access rules determine which I/O accesses to peripheral components, such as the sensor, actuator, RF module, off-chip memory, etc., are legal for each state. For example, micro pump access is legitimate only when the host microcontroller is in the Infuse insulin state, and only once per entrance to this state. The access pattern to the I/O components (I/O access data) is also examined by the rules. For example, ceaseless RF access attempts intended to prematurely drain the battery can be detected by limiting the RF module access frequency. I/O access rules do not define the range of actuator operations, i.e., the amount of insulin infusion, which is checked by physiological rules.

Physiological rules—as discussed above and shown in FIG. 2B, insulin infusion can be triggered through two modes: basal and bolus. Depending on which mode has triggered insulin infusion, the safe range of insulin amount is different. The program state reported by the host microcontroller, which is first checked by the state transition rules, is used to infer the mode of operation. Then, the BG levels (physiological state) obtained by monitoring the RF module activities is used for checking the physiological rules. The BG levels are used to verify if the insulin infusion command issued by the host microcontroller is safe or not, taking into account the inferred mode of operation.

In general the state transition rules and I/O access rules relate to the system's “internal” state (i.e., the device's state), the physiological rules pertain to the system's “external” state (i.e., user's state). The system disclosed herein makes more precise decisions by looking at both the internal and external state. In addition, some rule enforcements are performed not immediately after the occurrence of a device activity, but after some time has elapsed. A time-triggered rule is dynamically generated in the context of any of the three types of rules described above. If an expected state transition or I/O access is not observed by the safety coprocessor within a predefined time frame, it implies that the host microcontroller or peripheral components are not responding, which can be ascertained by time-triggered state transition rules or I/O access rules. Time-triggered physiological rules can be used to periodically monitor changes in the physiological status or after the actuator operation.

Follow-up Actions

Upon the detection of unsafe operations, the safety coprocessor performs necessary follow-up actions. In the case of violations of state transition rules or I/O access rules, the safety coprocessor may generate an output for the user interface to raise a user-perceptible warning so that the user can take necessary action, such as device checkup. The user interface may be implemented on an external device (e.g., a smartphone) to provide more details about the problem. If the violations are persistent and may damage the device (e.g., through premature battery drain), the safety coprocessor may temporarily disable and isolate the problematic components. It may also reset or turn off the host microcontroller, if needed, by shutting down the power to it or asserting the reset signal. In such cases, the safety coprocessor allows the execution of minimal medical functionality to give time to the patient to take necessary action. For example, in an insulin pump, a pre-programmed basal insulin is infused at a fixed rate, while bolus doses are manually infused using an emergency insulin pen until the problem is resolved by a medical professional. When a physiological rule is violated, in addition to blocking the suspicious operation, the patient can be asked to confirm the validity of the operation in a more trustworthy manner. For example, a wireless insulin infusion request out of the safe range can be displayed and confirmed using on-board user interfaces.

Prototype Implementation

In this section, a prototype design of a safety-enhanced controller board based is disclosed. Also a prototype insulin pump system is disclosed. This design incorporates the controller board and integrates the system with a human body model simulator to evaluate safety rule checking.

Insulin Pump System Prototype

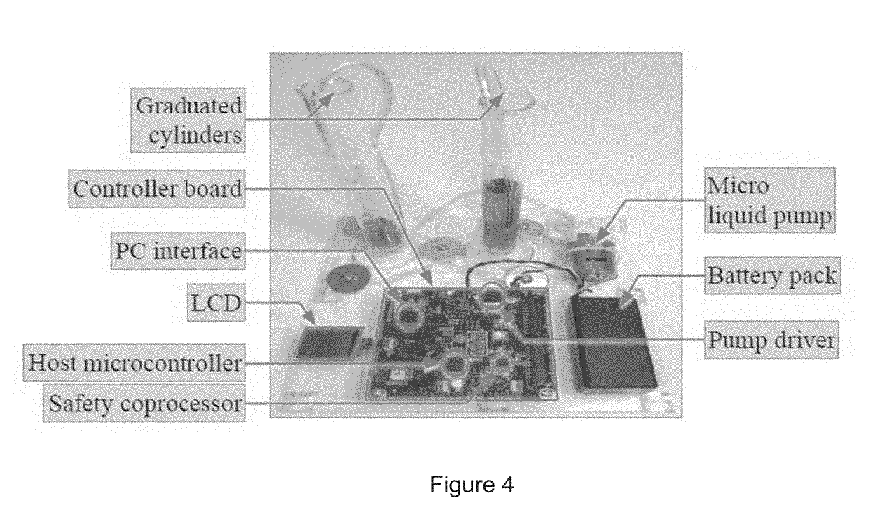

FIG. 4 shows a prototype insulin pump system. An EFM32WG from Silicon Labs is used to implement the host microcontroller, which incorporates an ARM Cortex M4 core. Its maximum frequency is 48 MHz and active mode power consumption is 225 A/MHz. The safety coprocessor is implemented with an emphasis on low sleep-mode power consumption because it is in the sleep mode most of the time, waiting for events to trigger safety rule checks. The safety coprocessor is implemented using an EFM32HG from Silicon Labs, based on an ARM Cortex MO+core, which consumes only 0.9 A in the deep sleep mode with the real-time clock (RTC) on. When in active mode, it consumes 114 A/MHz at up to 25 MHz. A low-energy universal asynchronous receiver/transmitter (LEUART) channel is used as the IMC channel between the two microcontrollers.

An LCD, a battery pack, and a micro liquid pump are integrated on the board to compose a complete insulin pump system. The micro liquid pump moves the liquid from one graduated cylinder to another. Note that this pump is not a precision pump used in actual insulin pumps but is only used for demonstration to visualize infusion of insulin using colored liquid. Instead of measuring the power consumption of this mock pump, the pump power consumption of the pump is estimated based on pump actuation.

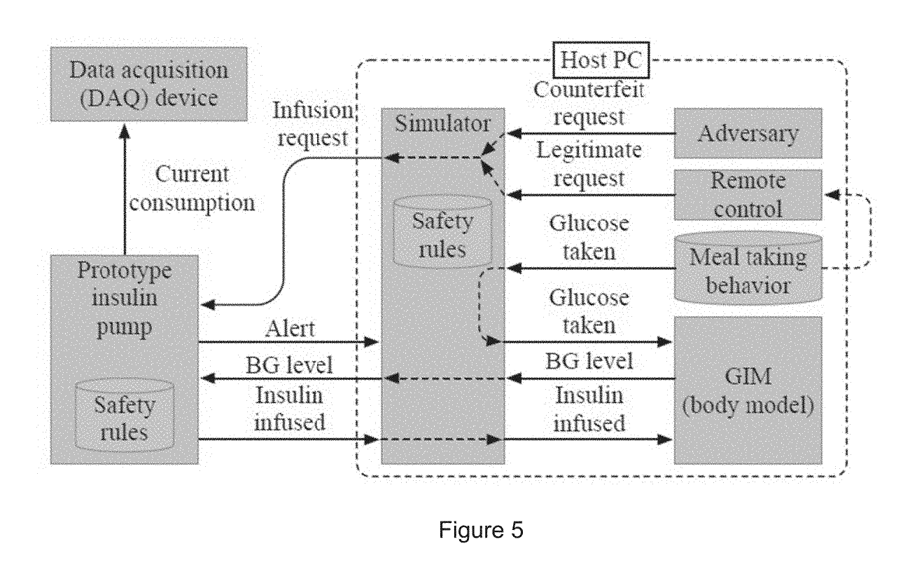

FIG. 5 shows a prototype insulin pump and human body model simulator integrated for safety rule development and evaluation running on a PC. On the PC side, MatLab software is used to simulate the physiological behavior of a diabetic patient. A well-known physiological model, called the Glucose-Insulin Model (GIM), simulates BG level variations based on glucose and insulin levels. The GIM is modified to interact with the controller board to update the BG levels and insulin infusions on the fly. The prototype insulin pump receives BG levels and insulin infusion requests from the simulator and performs necessary insulin infusion operations. The amount of insulin infused is notified to the simulator and, in turn, to the GIM, which updates the BG level. The adversary on the PC can generate counterfeit insulin infusion requests. The safety rules are implemented in the simulator as well, in order to verify the functionality of safety rule checking performed by the safety coprocessor.

Performance and Energy Overheads

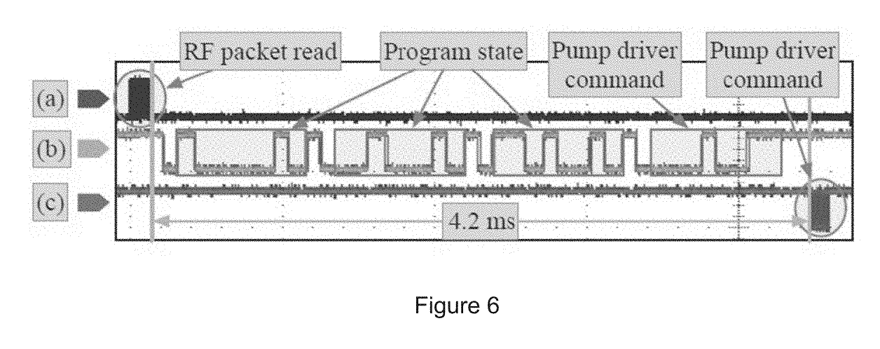

Since the modification in the host microcontroller firmware is mostly for IMC message generation, performance degradation is negligible. Nevertheless, by introducing a safety coprocessor, a propagation delay is introduced for actuator commands issued by the host microcontroller to reach the actuator. This delay is due to the time taken to receive the actuator command, check safety conformance, and re-issue the command to the actuator. FIG. 6 shows the communication waveform from receiving an RF packet to issuing a command to the pump driver. Waveform (a) is the SPI clock of the RF module. Waveform (b) shows there are three program transitions after receiving the RF packet before a pump driver command is issued by the host microcontroller. The safety of this command is checked and it is re-issued by the safety processor, as shown in Waveform (c). The time taken is 4.2 ms, but the majority of this time is spent on sending three state transition messages and one actuator command message over the IMC channel. The time spent on safety checking performed after receiving the actuator command message is almost negligible. The delay of 4.2 ms is not a concern for insulin infusions that may take up to a couple of seconds. In other IWMDs in which this time delay is not tolerable, a faster IMC channel can be used to reduce the delay to less than 1 ms.

The power consumed by the safety coprocessor was also measured. In IWMDs like insulin pumps, where the majority of energy is consumed by the peripheral components, such as the sensor, actuator, and RF module, the microcontrollers' power consumption is not significant. In the disclosed experiments, the safety coprocessor consumed less than 0.1 mA on an average. For an insulin pump running for one month with a 2500 mAh AA-sized battery, the safety coprocessor incurs just 3% energy overhead. Generally, safety rule checking is computationally much simpler than executing the medical functionality itself, hence a more low-power processor can be used. Moreover, the safety coprocessor can be more heavily duty-cycled than the host microcontroller because safety rule checking is triggered mainly by the host microcontroller's activities. As a result, the power consumption of the safety coprocessor scales with that of the host microcontroller in other IWMDs.

Patient Model

In this example, we assume a patient with Type 1 diabetes. The default parameters of the patient defined in the GIM are: a body weight of 78 kg, basal glucose dose of 180 mg/dL, and endogenous glucose production of 2.40 mg/kg/min. Breakfast, lunch, and dinner are assumed to be taken at 8:00 AM, 12:00 PM, and 8:00 PM, respectively, and 45 g, 70 g, and 70 g of glucose ingested at these times. To maintain a stable BG level, 3 units, 7 units, and 7 units of pre-meal bolus is assumed to be infused 20 minutes before each meal.

State Transition Rules

The state transition rules for this example are defined based on the state transition diagram shown in FIG. 2(b). State transitions are legal only if defined by the directed edges; all other transitions are considered a violation. We profile the execution time and define the time-triggered rules under the transition time constraint.

I/O access rules—these rules are also defined based on the same state transition diagram. Two peripheral components are monitored: the RF module and micro pump. The RF module can be accessed only from the RF access state, and the micro pump can be accessed only from the Insulin infusion state. If any peripheral component is accessed from other than these legal states, it is a violation of the I/O access rule.

Physiological rules—A physiological rule associated with the operation of the micro pump is defined. Whenever an insulin infusion command is issued to the micro pump for a pre-meal bolus dose, the safety coprocessor generates an instance of a time-triggered physiological rule. The details of this physiological rule and the unsafe situation prevented by this rule are described below.

Scenario 1: Wireless Attack Exploits Buffer Overflow

The first attack scenario is triggering an insulin overdose by exploiting buffer overflow. If the input data are deliberately designed to alter the program execution path, it may result in executing illegal, potentially harmful code. This kind of attack can be launched against embedded systems through wireless programming.

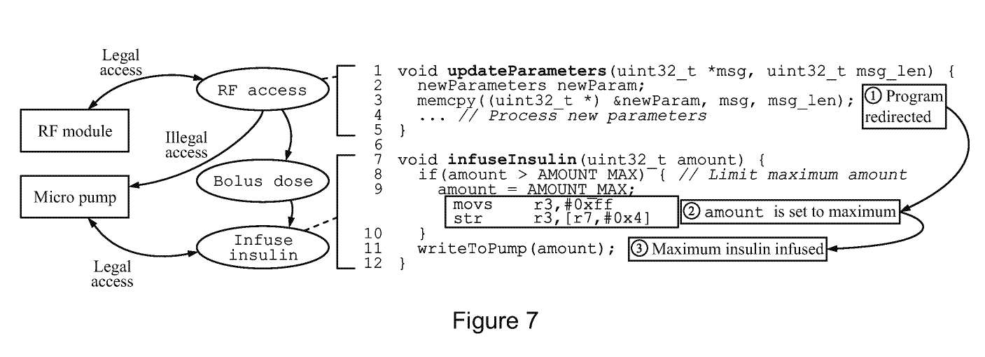

FIG. 7 shows a buffer overflow that triggers the micro pump to infuse the maximum amount of insulin. The variable r3 is the register for “amount.” In this example, the parameter reprogramming feature is exploited through the wireless channel. A snippet of the source code is shown in FIG. 7. The msg packet is received and transmitted to updateParameters( ) to update parameters. We introduce a hypothetical but very common vulnerability here by using memcpy( ) without boundary checking. In the infuseInsulin( ) function, the argument amount is checked and constrained to not exceed the maximum AMOUNT_MAX. Originally, the assignment statement of Line 9 is executed only when amount is greater than AMOUNT_MAX. However, if the program is redirected to Line 9 from outside this function, it can bypass the “if” clause and unconditionally assign AMOUNT_MAX to amount. This manipulated amount is transmitted to the writeTo Pump( ) function, which immediately infuses the excess amount of insulin. This attack executes a subroutine that is already present in the original code and thus does not need to inject attack code into the memory, which makes it difficult to defend against.

Defense

We can thwart this attack using the I/O access rule defined previously. The program is in the RF access state until the buffer overflow takes place through mempcy( ). It is then redirected to infuse

Insulin( ) without reporting program state transition to the safety coprocessor. When writeToPump( ) is called, the safety coprocessor discovers the anomaly that the micro pump is accessed from the RF access state. The I/O access rule restricts this access so that the micro pump is only accessed from the Infuse insulin state, as shown in FIG. 7. As a result, the micro pump driver command is blocked by the safety coprocessor, and does not reach the pump driver.

Note that this I/O access rule is not defined to specifically detect buffer overflow. Hence, any attacks or malfunctions that are manifested as similar I/O access violations will be detected by this rule. Also, this rule is orthogonal to conventional buffer overflow detection techniques.

Scenario 2: Bolus Infusion without Taking a Meal

As the second scenario, we consider the case wherein bolus insulin is infused through the normal procedure of the insulin pump, but the expected glucose is not ingested following the infusion.

Human Error/Attack Description

Bolus insulin should be infused when a meal is expected soon. In the absence of additional carbohydrate after the infusion, the excessive insulin may cause hypoglycemia. It is possible that the patient forgets about or delays a scheduled meal after infusing bolus insulin. Such an infusion could also be triggered by a malicious third party. It is possible to make the insulin pump infuse bolus insulin by creating a malicious control command that is not recognized as malicious. If this attack is launched when the user has no plan to take a meal or is unable to respond (e.g., when asleep), it may be successful in harming the patient.

It is difficult to distinguish an unsafe bolus insulin infusion from a safe one at the time of infusion. For example, a bolus insulin infusion is legal at the time of infusion if the patient originally had a plan to take a meal, but it later turns out to be unsafe when the meal is missed or delayed. For an unsafe operation that cannot be identified beforehand, the next line of defense is to minimize the harm as soon as possible by alerting the patient to the excessive insulin. Insulin overdose is typically treated by taking a skipped meal, sweets, glucose tablets, etc., if it is noticed early enough.

Defense

By using a time-triggered physiological rule, we can identify unsafe bolus insulin infusion. Based on the fact that bolus insulin infusion should be followed by additional carbohydrate intake, this rule checks if an increase in the BG level is observed a certain period of time after the bolus insulin is infused. Whenever bolus insulin is infused, an instance of this time-triggered rule is registered and checked later. In this experiment, the rule checks if the BG level has increased by more than 0 mg/dL, one hour after any amount of bolus insulin infusion. These values are empirically determined from the simulation results of the GIM. (In practice, they should be determined by the medical professionals based on the physiological characteristics of the patient.)

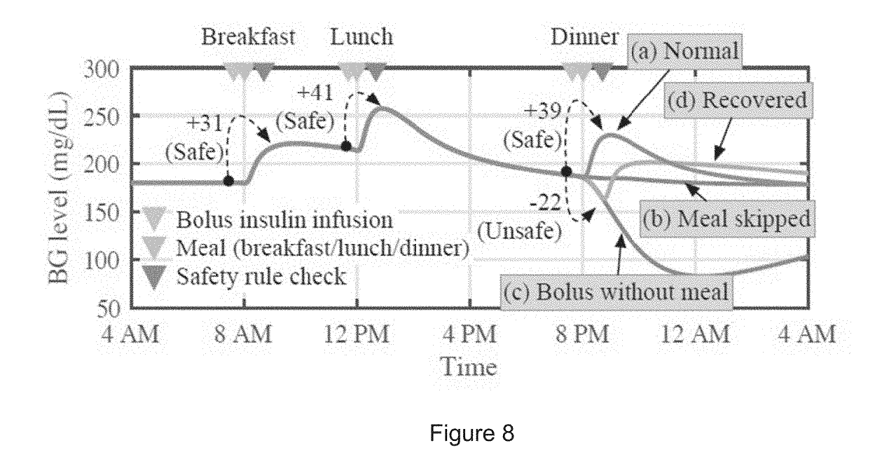

This simple rule is effective in detecting the aforementioned unsafe bolus insulin infusion. FIG. 8 shows BG level variation under various scenarios. Curve (a) shows the BG level variation in the normal case in which dinner (at 8:00 PM) and the pre-dinner bolus insulin (at 7:40 PM) are present as planned. Curve (b) denotes the case in which the patient did not have dinner, and the pre-dinner bolus insulin is not infused either. While this scenario is not recommended, it does not immediately harm the patient with low BG levels. However, if bolus insulin is infused without having dinner (due to human error or malicious attack), the BG level may drop significantly, as shown in Curve (c). The BG level drops to as low as 83 mg/dL around midnight. The safety coprocessor checks the physiological safety rule at 8:40 PM, detects the drop in BG level, and raises a warning about the violation. This prevents severe hypoglycemia by detecting unsafe operation at an early stage, as shown in Curve (d).

Note that this physiological rule is not aimed at any specific human error or security attack being the source of the unsafe operation. By enforcing a simple rule that checks the sensor input, triggered by a communication between the host microcontroller and actuator, it can successfully detect the potentially harmful consequence of the unsafe operation and inform the patient.

It should be understood that many variations are possible based on the disclosure herein. Although features and elements are described above in particular combinations, each feature or element can be used alone without the other features and elements or in various combinations with or without other features and elements. The digital processing techniques disclosed herein may be partially implemented in a computer program, software, or firmware incorporated in a computer-readable (non-transitory) storage medium for execution by a general-purpose computer or a processor. Examples of computer-readable storage mediums include a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs).

Suitable processors include, by way of example, a general-purpose processor, a special purpose processor, a conventional processor, a digital signal processor (DSP), a plurality of microprocessors, one or more microprocessors in association with a DSP core, a controller, a microcontroller, Application-Specific Integrated Circuits (ASICs), Field-Programmable Gate Arrays (FPGAs) circuits, any other type of integrated circuit (IC), and/or a state machine.

What is claimed is:

1. An implantable/wearable medical device configured for use with a plurality of sensors, the device comprising:
a host microcontroller, a safety coprocessor and an actuator, the host microcontroller being configured to receive physiological data from the sensors and generate actuator commands for the actuator, the host microcontroller being configured to generate program state data for transmission to the safety coprocessor,
the safety coprocessor being configured to receive the physiological data from the sensors and I/O access data and the program state information from the host microcontroller and determine whether there is a safety rule violation, the safety coprocessor being configured to issue the actuator command to the actuator if no safety rule violation is detected, the safety coprocessor being configured to initiate safety procedures if a safety rule violation is detected.

2. The medical device of claim 1 wherein the safety coprocessor is configured to perform safety rule checking based on state transition rules, I/O access rules and physiological rules.

3. The medical device of claim 2 wherein the state transition rules are based on the host microcontroller program state.

4. The medical device of claim 2 wherein the I/O access rules are based on access to I/O components.

5. The medical device of claim 2 wherein the physiological rules are based on physiological data received from the sensors.

6. The medical device of claim 2 wherein the physiological rules are based on a time lapse.

7. The medical device of claim 1 wherein the safety coprocessor is configured to communicate with a user interface to generate an alarm after a safety rule violation is detected.

8. The medical device of claim 1 wherein the safety coprocessor is configured to reset the host microcontroller after a safety rule violation is detected.

9. The medical device of claim 1 wherein the safety coprocessor is configured with a safety rule evaluation engine, a violation response engine and a steering logic engine.

10. The medical device of claim 8 wherein the safety rule evaluation engine is configured to receive the program state data from the host microcontroller, sensor data from sensors and actuator commands from the host microcontroller, the safety rule evaluation engine being configured to detect a safety rule violation and generate a rule violation status output and cut off output.

11. The medical device of claim 8 wherein the steering logic engine receives the cut off output from the safety rule evaluation engine and if no safety rule violation is detected, the actuator command is routed to the actuator, if a safety rule violation is detected the steering logic engine blocks the actuator command or sensor data from reaching the host microcontroller.

12. A method for detecting a safety rule violation in an implantable/wearable medical device configured for use with a plurality of sensors, the method comprising:
providing a host microcontroller, a safety coprocessor and an actuator, the host microcontroller being configured to receive physiological data from the sensors and generate actuator commands for the actuator, the host microcontroller being configured to generate program state data for transmission to the safety coprocessor,
the safety coprocessor being configured to receive the physiological data from the sensors and I/O access data and the program state information from the host microcontroller and determine whether there is a safety rule violation, the safety coprocessor being configured to issue the actuator command to the actuator if no safety rule violation is detected, the safety coprocessor being configured to initiate safety procedures if a safety rule violation is detected.

13. The method of claim 12 wherein the safety coprocessor is configured to perform safety rule checking based on state transition rules, I/O access rules and physiological rules.

14. The method of claim 13 wherein the state transition rules are based on the host microcontroller program state.

15. The method of claim 13 wherein the I/O access rules are based on access to I/O components.

16. The method of claim 13 wherein the physiological rules are based on physiological data received from the sensors.

17. The method of claim 13 wherein the physiological rules are based on a time lapse.

18. The method of claim 12 wherein the safety coprocessor is configured to communicate with a user interface to generate an alarm after a safety rule violation is detected.

19. The method of claim 12 wherein the safety coprocessor is configured to reset the host microcontroller after a safety rule violation is detected.

20. The method of claim 12 wherein the safety coprocessor is configured with a safety rule evaluation engine, a violation response engine and a steering logic engine.

21. The method of claim 20 wherein the safety rule evaluation engine is configured to receive the program state data from the host microcontroller, sensor data from sensors and actuator commands from the host microcontroller, the safety rule evaluation engine being configured to detect a safety rule violation and generate a rule violation status output and cut off output.

22. The method of claim 20 wherein the steering logic engine receives the cut off output from the safety rule evaluation engine and if no safety rule violation is detected, the actuator command is routed to the actuator, if a safety rule violation is detected the steering logic engine blocks the actuator command or sensor data from reaching the host microcontroller.
